(19) United States
(12) Patent Application Publication
(10) Pub. No.: US 2006/0155153 A1
(43) Pub. Date: Jul. 13, 2006

(54) Methods for making(-)-menthol and oxygenated menthane compounds

(76) Inventor: Sergey Selifonov, Plymouth, MI (US)

Correspondence Address: FISH & RICHARDSON P.C., PO BOX 1022, MINNEAPOLIS, MN, 55440-1022, US

(21) Appl. No.: 10/523,050

(22) PCT Filed: Jul. 24, 2003
(86) PCT No.: PCT/US03/23120

§ 371 Date: Sep. 22, 2005

(30) Foreign Application Priority Data
Aug. 2, 2002 (US) 60400454

(51) Int. Cl. C12P 7/12; C12P 7/02

(52) U.S. Cl. 568/829; 435/155

(57) ABSTRACT

Processes are described for the preparation of (−) menthol from 4R-(+)-menthene or from (+)-limonene, wherein the starting material is hydroxylated using a catalyst comprising at least on polypeptide to form trans-piperitol or trans-isopiperitenol, respectively, followed by hydrogenation to from (−)-menthol.

### CROSS-REFERENCE TO RELATED APPLICATIONS

The present application claims priority to and benefit of the U.S. provisional patent application No. 60/400,454, filed Aug. 2, 2002, entitled “Methods for making (−)-menthol and oxygenated menthane compounds” by Selifonov, S. A., which is incorporated herein by reference.

### TECHNICAL FIELD

The present invention relates to novel and improved methods of preparation of (−) menthol, and in particular, methods that use biological oxidation of 4R(+)-1-menthene to trans-piperitol (1R,6S-6-isopropyl-3-methylcyclohex-2-ene-1-ol), and/or 4R(+)-limonene to trans-isopiperitenol (1R,6S-6-isopropenyl-3-methylcyclohex-2-ene-1-ol). The resulting hydroxylated menthane derivatives are converted to (−)menthol using selective catalytic hydrogenation.

### BACKGROUND

(−)-Menthol (1R,2S,5R-2-isopropyl-5-methylcyclohexanol) is widely used in large quantities in the flavor industry to impart the characteristic cooling menthol taste in many consumer products including candies, chewing gums, toothpaste, mouthwash and tobacco articles. Consequently, many research groups have undertaken various approaches to develop efficient methods for making menthol from a variety of raw materials. Many methods for synthesis of (−)-menthol are known in the art. Among such methods are those that use various raw materials including myrcene, citronellal, (−)-beta-phellandrene, (−)-delta-3-carene, (+)-limonene, thymol, pulegone and (−)piperitone and piperitenone.

However known synthetic methods using these raw materials are too complex and expensive to make menthol on an industrial scale. For example, complex reactions with multiple steps are required to convert the raw materials to menthol. The raw materials are expensive, and expensive catalysts and reagents must be used in the reactions. In many cases the reagents and catalysts require special handling that is not suitable for cost efficient industrical production. The reactions produce menthol in low yields, and the menthol reaction products that result must be extensively purified to eliminate isomers and unwanted by-products.

As result of such deficiencies, synthetic (−)-menthol is quite expensive. Most commercially available (−) menthol is currently produced from agricultural sources such as essential oils of Mentha species typically represented by cornmint or peppermint. However, the agricultural production of these essential oils demands multi-year committals of large areas of land for mint plantations. Large amounts of fertilizers, herbicides and pesticides are typically used to achieve optimal yields and economic efficiency, and residual herbicides and pesticides often contaminate the resulting mint oils.

Menthol from agricultural mint oil production also varies considerably in quality and purity. Agricultural production of essential oils including (−)-menthol is also subject to adverse weather conditions, especially drought, and the production of (−)-menthol and its pricing fluctuates dramatically over time.

Biosynthesis of (−)-menthol in Mentha species is a complex multi-step process that currently understood only in part. However, the entire natural biosynthetic pathway for menthol biosynthesis is quite complex, hence development of genetically modified mint species with significantly enhanced (−)-menthol production via natural pathway is very difficult, costly and has proven elusive to date.

### SUMMARY

In one aspect, the invention is a method for making (−)menthol, the method including:
a) providing starting material including 4R(+)-1-menthene having formula (1): 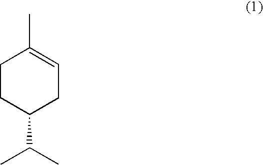
b) oxidizing the starting material in the presence of a catalyst including at least one polypeptide capable of hydroxylating at least one enantiomer of 1-menthene or limonene at an allylic carbon, thereby forming a hydroxylated menthene product including at least 50% of the trans-piperitol having formula (2): 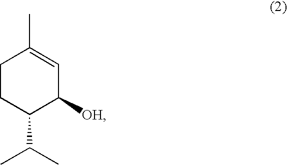
c) hydrogenating the trans-piperitol of formula (2) in the presence of a catalyst to form (−)-menthol having at least about 62% chemical purity, and at least about 90% enantiomeric excess, the (−)menthol represented by formula (3):

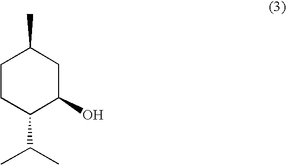

In another aspect, the invention is method for making (−)menthol, the method including:
a) providing starting material including 4R(+)-limonene having formula (4): 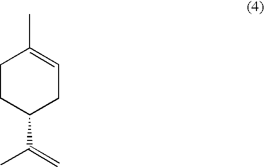
b) oxidizing the starting material in a presence of catalyst including at least one polypeptide capable of trans-hydroxylation of at least one enantiomer of 1-menthene or limonene at an allylic carbon, thereby forming a hydroxylated menthene product including at least about 80% of the trans-isopiperitenol having formula (5): 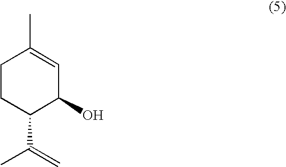
c) hydrogenating the trans-isopiperitenol of formula (5) in the presence of a catalyst, wherein hydrogenation selectivity of the catalyst provides (−)-menthol formation with a ratio of (−)menthol to (−)isomenthol of at least about 70:30, thereby forming (−)-menthol having at least about 70% chemical purity, and at least about 90% enantiomeric excess, said (−)menthol represented by formula (3):

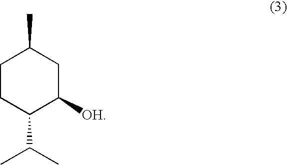

The methods described herein provide substantially pure (−)-menthol of high enantiomeric purity from inexpensive, renewable and abundant starting materials originating as by-products of the citrus industry. Other chemical synthesis methods for making (−)-menthol are long, complex, and give complex mixtures of products. Typically, conventional synthesis methods rely on use of more expensive raw materials, or of expensive catalysts that require meticulous handling. A combination of a selective biological oxidation step with a selective hydrogenation step provides an efficient two-step process. Improvements in the selectivity of the catalytic hydrogenation step described herein provide economic advantages over less selective methods by minimizing formation of low-value unwanted by-products, which hence offers cost advantages over existing menthol procurement methods.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION

In one aspect, the invention provides a process for making (−)-menthol from readily available 4R(+)-1-menthene having formula (1). This method includes two principal steps according to the following synthesis scheme:

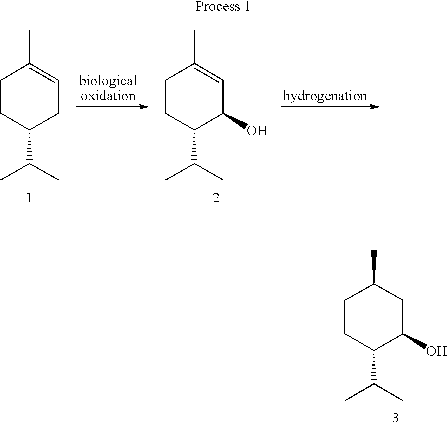

In another aspect, the invention also provides a process for making (−)menthol from 4R(+)limonene according to the two-step process shown in the following synthesis scheme:

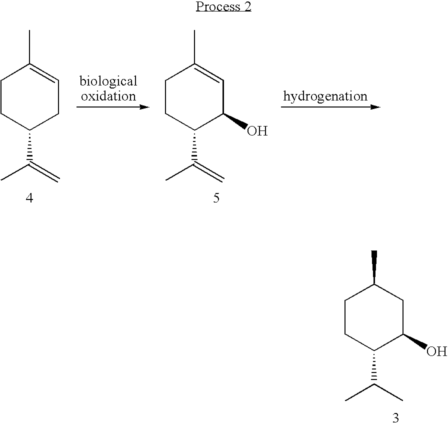

The biological oxidation steps of the inventive processes 1 and 2 above are highly selective and have an excellent rate of conversion for hydroxylation of (+)-1-menthene to trans-piperitol (2); and, for hydroxylation of (+)limonene (4) to trans-isopiperitenol (5).

In addition, the hydrogenation steps of the inventive processes 1 and 2 above have improved selectivity and produce fewer unwanted by-products as trans-piperitol (2) or trans-isopiperitenol (5) are converted to (−)-menthol (3).

The 4R(+)-1-menthene (Formula 1) of high optical purity (usually, with enantiomeric excess over 97%) is readily available by means of catalytic partial hydrogenation of 4R(+)-limonene, the principal ingredient of low-cost orange peel oil. The 4R(+)-1-menthene can be made, for example, using a Raney nickel catalyst or other ordinary hydrogenation catalysts such as nickel, palladium or platinum or their oxides, optionally immobilized on various supports such as carbon, silica, alumina, zirconia, alkali-earth metal carbonates, sulfates, phosphates and the like, and under conditions that are sufficient to reduce the exocyclic olefnic bond of limonene, but not the double bond in the cyclohexene ring. Ordinarily, such hydrogenation is carried out by adding about one molar equivalent of hydrogen in order to avoid overhydrogenation of (+)limonene to menthane.

Biological Hydroxylation.

The first reaction in processes 1 and 2 above is biological hydroxylation of 4S(+)-1-menthene and 4R(+)limonene, respectively. This biological reaction can be accomplished using polypeptide that is capable of hydroxylating either 4R(+)-1-menthene or 4R(+)-limonene at an allylic position C(3). For example, enzymes such as a limonene-3-hydroxylase or 1-menthene-3-hydroxylase can be used to hydroxylate 4R(+)-1-menthene or 4R(+)-limonene, respectively. Specifically, in process 1, 4S(+)-1-menthene is hydroxylated to produce the trans-hydroxylated product of formula (2), (1R,6S-6-isopropyl-3-methylcyclohex-2-ene-1-ol, (trans-piperitol).

Many enzymes can be selected to conduct this hydroxylation step in processes 1 and 2. For example, U.S. Pat. Nos. 6,083,731 and 6,194,185 describe suitable enzymes from mint and methods for isolating enzymes with limonene-3-hydroxylase activity from other species. (GenBank accession numbers: AR134847, AR134848, AR134849, AR134850). The 4S(−)limonene-3-hydroxylases from mint are capable of 3-hydroxylating substrates of opposite chiral series such limonene 4R(+)-limonene and 4R(+)-1-menthene (F. Karp, et al, Archives of Biochemistry and Biophysics, 1990, 276(1):219-226) and thus are useful for conducting the biological hydroxylation step in the processes described herein.

Limonene hydroxylases that hydroxylate this substrate at positions other than C(3) can be modified by mutagenesis to aqcuire 3-trans-hydroxylating ability. For example, M. Schalk and R. Croteau (Proc. Natl. Acad. Sci., 2000, 97(22):11948-11953) have reported that 4S(−)-limonene-6-hydroxylase can be converted to a 4S(−)-limonene-3-hydroxylase by a single aminoacid substitution. Such mutant enzymes are suitable to carry out conversion of R(+)-1-menthene or R(+)limonene to the 3-trans-hydroxylated products.

Examples of species that contain enzymes and nucleic acids encoding them include species from family Lamiaceae, such as Ocimum (basil), Lavandula (lavender), Origanum (oregano), Mentha (mint), Salvia (sage), Rosmecinus (rosemary), Thymus (thyme), Satureja and Monarda. Other species include those from family Umbelliferae, including, but not limited to, the following species: Carum (caraway), Anethum (dill), Feniculum (fennel) and Daucus (carrot). Other species also include those from family Asteraceae (Compositae), including, Artemisia (tarragon, sage brush), Tanacetum (tansy). Families such as Rutaceae, Rosaceae, Myrtaceae (e.g., eucalyptus, and Eucalyptus dives in particular), Gramineae, Geranaceae (Geraniurn) and certain conifers. U.S. Pat. Nos. 6,083,731 and 6,194,185 also describe certain methods that are useful for isolating nucleic acids encoding limonene 3-hydroxylase from the above referenced organisms. The mint limonene hydroxylases can be functionally expressed in microorganisms, as described by C. Haudenschild et al, (Archives of Biochemistry and Biophysics, 379(1):117-136, 2000).

Further examples of polypeptides having limonene-3-hydroxylase activity are enzymes that can be found in certain microorganisms, such as bacteria, yeast and fungi.

Mutant cytochrome P450 enzymes derived from camphor-5-hydroxylase, and in particular, the F87W-Y96F-V247L mutant (such as described by S. G. Bell, R. J. Sowden and L.-L. Wong, 2001, Chemical Communications, p. 635-636, and in the International PCT Application No. WO 00/03273, by L.-L. Wong, et. al, published on Jun. 2, 2000), are also suitable to carry out conversion of R(+)-1-menthene or R(+)limonene to the 3-trans-hydroxylated products.

Examples of fungi having limonene-3-hydroxylase activity include, for example, Aspergillus fumigatus ATCC1028 (formerly, A. cellulosae M-77), which displayed a low level of limonene-3-hydroxylase activity along with limonene-6-hydroxylase activity (Y. Noma, et al, 1992, Phytochemistry, 31(8):2725-2727).

M. S. van Dyk et al, (Biotechnology Letters, 1998, 20(4): 431-436) have described a strain of black yeast Hormonema sp. Y-0067 that forms practically pure trans-isopiperitenol (5) from (+)limonene.

One of ordinary skill in the art can readily isolate many microorganisms having limonene-3-hydroxylase or 1-menthene-3-hydroxylase activity from environmental samples. Nucleic acids from various environmental samples such as soils, sediments, surface and ground waters, sludges from sewage and chemical waste treatment facilities can be isolated directly by methods known in the art, and expressed in various prokaryotic or eukaryotic organisms, and the organisms can be screened for the desired limonene-3-hydroxylase or 1-menthene-3-hydroxylase activity.

Enzymes having limonene hydroxylating activity can be also found in such common classes of hemoprotein enzymes such as cytochrome P450 oxygenases, which currently include over 1400 enzymes from various organisms, as well as among iron-sulfur monooxygenases and aromatic ring dioxygenases that are capable of allylic monooxygenation of certain alkenes. The extent to which any of these enzymes are capable of 3-hydroxylation of limonene or menthene enantiomers in comparison with hydroxylation at other carbon atoms varies greatly, depending on the source of the enzymes and the nucleic acid sequences that encode the polypeptides.

In practice, various oxygenase enzymes can be screened for cis- or trans-3-hydroxylase activity and for other hydroxylating activities by incubating samples comprising such enzymes with limonene or 1-menthene under conditions suitable for detection of oxygenase activity for sufficient time, and the resulting products can be extracted and analyzed by various analytical methods for the presence of any oxidation products and for the presence of the desired 3-hydroxylated products in particular. Such methods typically include gas chromatography or high-pressure liquid chromatography, or these methods in conjunction with mass spectrometry.

In practice, it is useful to use simple pre-screening chemical methods for detection of hydroxylated products from limonene or 1-menthene, prior to conducting chromatography analysis, because such methods are less expensive and less time-consuming than the chromatography-based methods above, and also allow the worker to sort out inactive or less active enzymes. Typically, samples containing unknown amounts of at least one hydroxylated limonene or 1-menthene product can be recognized as positive by the oxidation of the biologically formed alcohols to corresponding carbonyl compounds comprising ketones and aldehydes. Oxidation of the hydroxylated limonene or menthene products to the corresponding carbonyl compounds can be carried out chemically or enzymatically. Methods for chemical oxidation of secondary or primary alcohols to corresponding carbonyl compounds without co-oxidation of olefinic bonds are well know in the art and many such ordinary methods can be used. Non-limiting examples of suitable reagents include pyridinium chlorochromate, chromium trioxide-pyridine, manganese dioxide, Dess-Martin periodinane, silver carbonate, and like. Biological oxidation can be carried out in the presence of a suitable alcohol dehydrogenase and cofactors, and can be readily accomplished by adding a suitable enzyme preparation or by co-expressing oxygenases of interest for screening for limonene hydroxylase activity in a suitable microbial host organism possessing alcohol dehydrogenase activity.

The resulting samples of reaction mixtures containing unknown carbonyl compounds derived from limonene or 1-menthene can be recognized as positive for the presence of carbonyl compound by reacting with hydrazine compounds, such as dinitrophenylhydrazine and like. Carbonyl compounds form various brightly colored hydrazone adducts with hydrazines, and thus enzymes having produced appreciable quantities of hydroxylated products from limonene or 1-menthene can be readily identified in the enzyme screening.

Regardless of the source of enzymes having appreciable limonene hydroxylating activity, in practice it is often necessary to improve properties of enzymes found in nature in respect to producing the trans-piperitol (2) from 4R(+)-1-menthene (1) and/or the trans-isopiperitenol (5) from (+)limonene (4), with satisfactory yield, rate and purity. Many methods for such improvements are known in the art, and they typically involve modifications of sequences of nucleic acids encoding polypeptides with limonene hydroxylating activity, thereby producing variants of modified nucleic acids that can be expressed to produce enzymes and the enzymes can be screened for their improved ability to hydoxylate 4R(+)-1-menthene at the carbon atom C(3) to form the trans-piperitol of formula (2), and/or to hydoxylate 4R(+)-limonene at the carbon atom C(3) to form the trans-isopiperitenol of formula (5). As used herein, the term “polypeptide” refers to a chain of amino acid residues of any length that has limonene 3-hydroxylase or 1-menthene 3-hydroxylase activity. Such methods include various mutagenesis and recombination techniques that allow for creating a number of variants of limonene hydroxylating enzymes.

Non-limiting examples of such representative methods include methods described in the following publications: Ho, S. N. et al, (1989, GENE, 77: 51-59), Site-directed Mutagenesis by overlap extension using the polymerase chain reaction; Horton, R. M. et al, (1989, GENE, 77:61-68), Engineering hybrid genes without the use of restriction enzymes: gene splicing by overlap extension; Berger et al, (1993, Anal. Biochem., 214:571-579), Phoenix Mutagenesis: One-step Reassembly of Multiply Cleaved Plasmids with Mixtures of Mutant and Wild-Type Fragments; Dillon and Rosen (1990, Biotechniques, 9(3):298-300), Prodromou and Pearl (1992, Protein Engineering, 5(8):827-829); PCT application No. WO0009682, U.S. Patent Application No. 20020183934, Methods for making character strings, polynucleotides and polypeptides having desired characteristics, by Selifonov, et. al. (2002); U.S. Pat. No. 6,352,859; U.S. Pat. No. 5,837,458; U.S. Pat. No. 6,171,820, and U.S. Pat. No. 6,440,668.

Random mutagenesis, site-directed mutagenesis, saturation mutagenesis, combinatorial gene library synthesis, gene shuffling in vitro and in vivo, error-prone polymerase chain reaction are all methods known in the art that allow for the creation of variants of limonene hydroxylating enzymes. In the present invention, such methods are recognized as part of directed evolution techniques that allow limonene 3-hydroxylase or 1-menthene 3-hydroxylase activity to be created in naturally occurring oxygenase enzymes that do not display such activity or to be enhanced in enzymes that have such activity. The processes of the invention is not limited to use of any particular directed evolution or mutagenesis methods, or variations on part of such methods. The above references to such methods are not intended to be limiting and are provided herein solely to illustrate various ways that a suitable or improved biological catalyst (i.e., enzyme) could be produced for the production of (−)-menthol.

Such methods can be used to obtain limonene 3-hydroxylase or 1-menthene 3-hydroxylase activity in enzymes that naturally are capable of hydroxylating other compounds, including, for example, cyclic terpenoid camphor, fenchone, 1,4-cineole, 1,8-cineole and other bicyclic terpenoids. Some examples of such methods are described in detail, for example for making cytochrome P450 enzyme mutants from camphor-5-hydroxylase, and in principle, from many cytochromes of P450 type, in PCT Application No. WO 00/03273.

Screening for improved characteristics that are beneficial to production of the trans-piperitol of formula (2) can be accomplished by several methods. For the purpose of practicing the present invention, the ability of an enzyme to produce trans-piperitol with high yields, high selectivity and high rates can be measured by analytical methods described above for use in initial screening for 3-hydroxylating enzymes acting on limonene or 1-menthene. Other desired characteristics that can be used solely or in conjunction with detection of production of the trans-piperitol (2) and/or the trans-isopiperitenol (5) are enzyme stability under the intended conditions for production and the ability of the enzyme to be produced satisfactorily in a microbial host organism that is suitable for industrial use or an organism related to such host.

The improved variants of limonene-3-hydroxylase or 1-menthene 3-hydroxylase can also be subsequently used to obtain additional improvements in an iterative process wherein any of the above mentioned techniques for sequence modification is applied one or more times, or different methods used sequentially, until the optimum activity is obtained.

For the purpose of production of (−)menthol according to the methods of the present invention, one or more enzymes having 4R(+)-1-menthene 3-hydroxylase activity can be recombinantly produced in a suitable host (e.g., prokaryotic or eukaryotic). The preferred host organisms are bacteria (including cyanobacteria), or fungi (including yeast or yeast-like fungi). Methods for expressing polypeptides in host cells are known in the art. One or more copies of nucleic acids encoding the hydroxylase polypeptides, and any reductase or other electron transfer-proteins, or their natural or artificial fusion arrangements can be expressed on different vectors that can be effectively maintained in a suitable host, or the nucleic acids can be integrated into host chromosomes. The present invention is not limited by use of a polypeptide that is based on a particular host, or of a vector, or of a promoter, or of specific gene arrangement, and one of ordinary skill in the art can introduce many variations on the part of such features of the polypeptide suitable for 3-hydroxylation of 1-menthene or limonene enantiomers.

In one embodiment of the present invention, the oxidation of 4R(+)-1-menthene is carried out using a cell free system, wherein 4R(+)-1-menthene 3-hydroxylase is selected from a class of enzymes known in the art as cytochromes P₄₅₀. In this embodiment, the oxidation reaction preferably is carried in the presence of an effective amount of hydrogen peroxide which is added gradually to the stirred reaction mixture comprising enzyme preparation, 1-menthene and aqueous buffer. Typically, about 1 equivalent of hydrogen peroxide is used to obtain 1 equivalent of hydroxylated 4R(+)-1-menthene products comprising trans-piperitol of formula (2). The enzyme for use in this embodiment can be purified, or used as crude microbial cell lysate, or as permeabilized microbial cells, or in immobilized form. The enzyme performance, such as reaction rate and selectivity, with concomitant improvements in acceptance of hydrogen peroxide by enzyme for the oxidation, and improvements in enzyme stability in the presence of hydrogen peroxide, can be attained by methods known in the art, for example, by methods described by Joo H., et al, (1999, Nature, 399(6737):670-673, Laboratory evolution of peroxide-mediated cytochrome P450 hydroxylation) and in the references cited therein, as well as by the directed evolution methods referenced above.

Hydroxylation of (+)limonene or (+)1-menthene can also be carried out using an isolated monooxygenase and an electroenzymatic reactor according to the methods known in the art, for example, U.S. Pat. No. 6,306,280. Such methods can be used as a suitable embodiment to practice the present invention in respect to carrying out selective biological enzyme-catalyzed 3-trans-oxidation of limonene or menthene, wherein the electroenzymatic reactor comprises one or more monooxygenase enzymes, such as hydroxylases from sources referenced herein, or mutant or modified hydroxylases according to methods referenced herein.

In another embodiment of the present invention, the oxidation of 4R(+)-menthene or 4R(+)limonene, or a mixture thereof, is carried out with substantially intact cells (whole cells) of microorganisms that possess sufficient 4R(+)-1-menthene or 4R(+)limonene 3-hydroxylase activity due to expression of at least one nucleic acid encoding 4R(+)-1-menthene 3-hydroxylase or limonene-3-hydroxylase. In this embodiment, the reaction is typically carried out in the presence of a sufficient amount of buffered aqueous medium with pH in the range between about 5 to 8. The oxidant in such reaction is preferably air or oxygen-enriched air, that is dispersed into reaction medium, and the reaction is carried out in a stirred reactor or fermentor. Additional nutrients can optionally be added to the reaction to provide for maintenance of the enzymatic activity and cofactor regeneration for the oxidation reaction. Examples of such nutrients include innocuous ordinary low-cost carbon sources such as glucose and other carbohydrates, glycerol, ethanol and like.

For the purpose of obtaining higher yields of hydroxylated products comprising trans-piperitol of formula (2) and/or trans-isopiperitenol of formula (5), and for simplifying product recovery after completion of the reaction, it is often advantageous to operate the reactor in a biphasic organic-aqueous mode wherein 1-menthene and/or (+)limonene is added to the reaction mixture as neat organic phase or in a mixture with other hydrocarbon or ether solvents. The hydrocarbons and ethers are preferably saturated, water-immiscible and selected from those compounds that have boiling points different from those of menthene, or of limonene, and of the hydroxylated products such as (2) and (5), by at least about 5 degrees Celsius. It is also preferred that hydrocarbons with low flash point temperatures are avoided for safety reasons. Because menthene and limonene are liquid compounds under ordinary temperatures for biocatalyst operation, menthene or limonene or mixtures thereof can be used themselves as organic solvents to create such biphasic organic-aqueous system. The present invention is not limited to use of a particular solvent for carrying out the biological oxidation steps in the process for making (−)menthol. The above examples are provided herein solely for the purpose of illustration of embodiments of the invention.

Using the biphasic system for oxidation of 4R(+)-1-menthene or (+)limonene requires that the biocatalyst can operate in the presence of the organic solvent or an aqueous emulsion thereof, whether the reaction is carried out using hydrogen peroxide or air or oxygen as oxidant, or using an electrochemical reactor. 4R(+)-1-Menthene or (+)limonene 3-hydroxylases having satisfactory solvent tolerance can be obtained by methods referenced above for modification of nucleic acids encoding such enzymes and having variants of the enzyme screened for ability to produce the desired hydroxylated products from 1-menthene in the presence of the organic solvent selected for carrying out menthene hydroxylation on industrial scale. For an embodiment of the invention wherein a reaction is carried out with whole microbial cells comprising a substantial proportion of viable cells, it is also advantageous to use host microorganisms that are naturally tolerant to high concentrations of 1-menthene, and/or of limonene, and of the hydroxylated menthenes such as (2) and (5), and, where applicable, of another organic co-solvent used. Organisms tolerant to high concentrations of hydrocarbons, and of limonene in particular, are known in the art; they can be used as examples of host organisms for expression of at least one nucleic acid having 4R(+)-1-menthene or (+)limonene-3-hydroxylase activity. For example, U.S. Pat. No. 5,763,237, and No. 5,652,137 describe strains of Bacillus stearothermophilus ATCC 55596, methods for isolation and use of B. stearothermophilus in production of certain oxygenated products from limonene, such as perillyl alcohol and carvone, but not the 3-oxygenated menthene or limonene compounds. While these patents are silent regarding possible uses of this organism for oxidation of 4R(+)-1-menthene using biphasic organic-aqueous system, it is the finding of the present invention, that such organisms can be used to oxidize menthene under conditions similar to those described for limonene oxidation in the referenced patents. However, the present invention is not limited to use of a particular solvent-tolerant microorganism to carry out hydroxylation of 4R(+)-1-menthene. 4R(+)-1-menthene tolerant microorganisms can be readily isolated by one of ordinary skill in the art using methods known in the art. For example, such microorganisms can be obtained by enrichment cultures that are established using for inoculum of the cultures samples of soils or sediments or sludges from various locations, and preferably, locations that have been exposed to significant discharges of hydrocarbons, limonene or essential oils, or from rotting piles of orange peel, or from the bottom of the storage tanks for petroleum products, or limonene, or citrus oil. Such storage vessels ordinarily contain minor amounts of water accumulated at bottom, and the samples of such water are useful for isolating microorganisms tolerant to hydrcarbons, limonene or menthene. To obtain menthene, limonene, terpene alcohol and/or hydrocarbon tolerant microorganisms, such enrichment cultures are typically established in ordinary shake flasks or fermentors or similar agitated or shaken vessels, using a biphasic organic-aqueous mixture comprising compounds of interest for which high microbial tolerance is desired, and an ordinary bacteriological aqueous medium, preferably supplemented with carbon sources, such as glucose, or ethanol or organic acids, or glycerol or aromatic hydrocarbons or aromatic carboxylic acids. After establishing microbial growth under such conditions, and typically, after a series of subsequent transfers to fresh biphasic system, pure microbial cultures comprising solvent-tolerant microorganisms can be obtained by dilution techniques or by plating on agar plates. Such organisms are preferred hosts for expression of the menthene- and limonene-hydroxylating enzymes used in the process of present invention.

Biological hydroxylation of limonene and menthene can be carried out in a broad range of temperatures, typically in the range between about 5° C. and about 100° C., and preferably between 15° C. and about 60° C. The process can be carried out at the temperatures outside of this range. However, at lower temperatures, the rate of biological hydroxylation is too slow, and at higher temperatures rapid inactivation of biocatalyst occurs.

The hydroxylated 4R(+)-menthene products comprising trans-piperitol of formula (2) are typically recovered by extraction after sufficient product concentration has been accumulated or, in the case of biphasic organic-aqueous reaction, by distillation of the organic phase from periodic withdrawal of organic phase from the reactor. In the latter case, the unreacted recovered menthene or limonene can be returned to the reactor for further biooxidation. The alcohols (2) and/or (5) can further be purified by distillation or crystallization to separate any other oxygenated by-products, if formed, or they can be subjected to the next step without substantial purification. In particular, when high purity (2) and (5) are formed in the biological step, for examples, when using highly selective catalyst such as Hormonema sp. Y-0067 yeast, purification of the terpene alcohols is not required.

Hydrogenation.

The second reaction in the method for making (−)menthol according to processes 1 and 2 is stereoselective hydrogenation of the trans-piperitol (2) and the trans-isopiperitenol (5), respectively, to the (−)-menthol of formula (3). Such reaction can ordinarily be carried out by methods known in the art, without stereoselectivity, or with a moderate stereoselectivity with the ratio of (−)-menthol to (−)isomenthol of up to about 75:25.

It has been found in the present invention that use of palladium or palladium oxide catalyst immobilized on calcium carbonate (Pd/CaCO₃ or PdO/CaCO₃, wherein the latter compound is ordinarily reduced to the former by hydrogen under typical hydrogenation conditions) offers significant advantages over coventional palladium and platinum-containing catalysts. The Pd/CaCO₃ or PdO/CaCO₃ catalysts attain a high product ratio that favors formation of (−)menthol (3) from trans-piperitol (2) in process 1 and from trans-isopiperitenol (5) in process 2. The Pd/CaCO₃ or PdO/CaCO₃ catalysts also minimize quantities of undesired by-products such as (−)-isomenthol, thereby offering greater selectivity than selectivities found for Pd/C and for PtO₂ (Adam's catalyst).

The latter catalyst (PtO₂) tested under broad range of solvents and other conditions and, as demonstrated by examples provided herein, is less selective than the palladium catalysts on either carbon or CaCO₃ supports. For example the best observed selectivities for (−)-menthol formation with PtO₂ catalyst is about 62% for hydrogenation of compound (2) and about 60% for hydrogenation of compound (5). In comparison, the corresponding selectivities for Pd/C catalyst are about 75% and about 70%. Further in comparision, the corresponding selectivites for Pd/CaCO₃ catalyst are about 85% and 79%.

Hydrogenation of trans-piperitol (2) to (−)-menthol (3) in process 1 proceeds more selectively and with lesser amounts of by-products than hydrogenation of trans-isopiperitenol (5) in process 2. Therefore, process 1, which uses a starting material including 4R(+)-1-menthene (1), or mixtures of predominant amounts of 4R(+)-1-menthene (1) with (+)limonene (4), is preferred over process 2 for industrial production of (−)menthol (3).

The (−)menthol (3) formed by hydrogenation of trans-piperitol (2) in process 1 is present in very high enantiomeric excess (over 99%), while the (−)menthol (3) formed in process 2 by hydrogenation of trans-isopiperitenol (5) over palladium catalysts was present in enantiomeric excess of about 94-96%. Therefore, from an industrial standpoint, the process for making menthol via trans-piperitol (2) in process 1 is preferred over the process variation via trans-isopiperitenol (5) in process 2, as separation of undesired (+)menthol enantiomer is a laboriuous and costly process.

Process 1, which includes biological 3-hydroxylation of (+)1-menthene (1), followed by hydrogenation of trans-piperitol (2) to (−)menthol, has a number advantages over the alternative method in process 2 based on 3-hydroxylation of (+)limonene (4), followed by hydrogenation of trans-isopiperitenol (5) to (−)menthol.

The trans-isopiperitenol of formula (5) is simultaneously allylic and homoallylic alcohol and hence is more susceptible than trans-piperitol of formula (2) to catalytic rearrangements during catalytic hydrogenation. For example, Leffingwell and Shackelford (Cosmetics and Perfumery, 89(6)68-89,1974), has reported yield of(−)menthol of unknown enantiomeric excess from hydrogenation of trans-isopiperitenol (5) “over 70%” using Pd/C catalyst. The results of Examples 13-20 below show that in fact, this catalyst in various solvents produces up to about 71% of (−)menthol by hydrogenation of (5), with about 24% of isomenthol, and the remainder being principally menthone isomers, pulegol and pulegone. In comparison, hydrogenation of trans-piperitol (2) using the same catalyst leads to the formation of up to 75% of menthol (when using ethanol as a solvent). That means significantly more of isomenthol by-product is formed during hydrogenation of (5) than during hydrogenation of (2), imposing significant costs associated with inefficient utilization of capacity for making unwanted compounds, and with product purification and by-product disposal.

In addition, biological hydroxylation of R(+)limonene to trans-isopiperitenol (5) by whole microbial cells is often accompanied by the formation of small amounts of the piperitenone that easily racemizes under aqueous conditions and is also prone to rearrangements due to migration of the homoallylic double bond in the isopropenyl side chain. Another advantage of process 1 using 4R(+)-1-menthene versus the (+) limonene in process 2 for the biological hydroxylation step is that menthene is more stable to spontaneous air oxidation and is less toxic to microorganisms than limonene. Limonene readily forms traces of epoxy and peroxy compounds that are inhibitory to microorganisms.

Stereoselective hydrogenation of trans-piperitol (2) in process 1 or trans-isopiperitenol (5) in process 2 can be carried out under a broad range of conditions. The hydrogenation is carried out typically in the presence of suitable solvent, such as alcohols, glycols, polyols and water or mixtures thereof. However, the hydrogenation reaction can also be carried without solvent, using neat (2) or (5) or mixtures thereof. Typically, alcohol or glycol or polyol have linear or branched or cyclic alkyl or alkyloxyalkyl chain having from 1 to 20 carbon atoms.

These solvents can be used in combination with other solvents such as hydrocarbons, chlorinated hydrocarbons, carboxylic esters and ethers. Such modifications are fully within the scope of the present invention. Hydrogenations in ethanol or water-alcohol mixtures, or without solvent, are preferred.

Hydrogenations in solvents including esters of monohydric alcohols and carboxylic acids, such as ethyl acetate, methyl prorionate, ispropyl acetate, diethyl succinate and the like, typically give lower stereoselectivity and lower (−)menthol yields, as compared to hydrogenations in alcohols, water, gycols, polyols and mixtures thereof.

Hydrogenation can be carried out in a broad range of hydrogen pressures. Hydrogenation can be carried out at atmospheric pressure or at increased pressure, typically between 1 and 100 atm. Hydrogenation at an elevated pressure, even at moderate pressures of about 3-5 atm results in significantly lower amounts of isomerization by-products such as menthones in the resulting product mixture. Hydrogenation can be carried out using hydrogen or mixtures of hydrogen and an inert gas, such as nitrogen or argon. Hydrogenation can also be carried out in the presence of other compounds such as sodium formate. Hydrogenation with substantially pure hydrogen is preferred.

Hydrogenation can be carried out over a broad temperature range, typically between 0° C. and 250° C. Reactions can also be carried out outside of this range. However, at lower temperatures the reaction is too slow, and at higher temperatures more of the unwanted by-products are formed. Reactions at the lower end of the specified temperature range tend to produce less of the isomerization byproducts such as menthone isomers. Reactions at the higher end of the specified range tend to produce more of isomerization and hydrogenolysis by-products (e.g. menthanes and menthenes). Temperatures in the range between about 20° C. and 220° C. are preferred.

The amount of catalyst required for carrying out the hydrogenation step is typically in the range from 0.01 to 20 molar % with respect to the amount of terpene alcohols (2) or (5). The reaction can be carried out with catalyst amounts outside of this range. However, with low catalyst amounts, the reaction is too slow, and high catalyst amounts incur higher capital and operating costs.

The (−)menthol (3) product can be purified by methods known in the art. Typically, such methods include distillation and/or crystallization, including crystallization of high purity (−)menthol by refrigeration of crude hydrogenation product in a process akin to purification for (−)menthol from cornmint (Mentha arvensis) or peppermint (Mentha piperita) essential oils widely practiced in the industry.

The invention has been described in preferred and exemplary embodiments and aspects, but is not limited thereto. Persons skilled in the art will appreciate that other modifications and applications fall within the scope of the invention. When the term “about” is used in the specification and claims in connection with a range of numbers, it is intended to modify both the low value and the high value of the range.

### EXAMPLES 1-12

Hydrogenations of trans-piperitol and trans-isopiperitenol using Adam's Catalyst (PtO₂).

99.4% pure trans-piperitol (2), with enantiomeric purity >99.5%, and 98.7% pure trans-isopiperitenol (5) with enantiomeric purity >98%, were obtained by biological hydroxylation of (+)menthene or (+)limonene, correspondingly, using a general biooxidation procedure published by M. S. van Dyk et al (1998, supra), with exception that gradual dropwise addition of the substrates was carried out over 12 hours, and high density cell cultures with apparent OD₆₀₀ in the range from about 50 to 200 were used. The hydroxylated compounds were extracted with ethyl acetate, compounds were recrystallized from hexane prior use. Weighed amounts of each terpene alcohols we taken for individual evaluation of various hydrogenation catalysts, solvents, temperatures and other conditions. The weighed amount of catalyst and the solvent were added to the hydrogenation vessel equipped with magnetic stirrer, and the whole was purged with hydrogen for 30 min to allow for complete reduction of catalysts. Reactions were started by adding trans-piperitol (2) or trans-isopiperitenol (5). For testing hydrogenation catalyst selectivity in water, 10% of methanol was added to facilitate solubility of starting materials and products. Catalysts were evaluated at calculated concentrations in the range of 0.1-20% mol/mol of Pd or Pt ratio to the terpene alcohols. Aliquots were periodically taken for GC and GC-MS analysis to evaluate reaction course, product formation and identity. The reactions were carried out until starting material was no longer detectable by GC and GC-MS. Typical results are summarized in the Table 1.

TABLE 1

| | | | Solvent | | Temperature, | Menthol | Menthol + |
|---|---|---|---|---|---|---|---|
| Example | Starting | Catalyst, | and | Reaction | ° C., and | to Isomenthol | isomenthol in |
| No. | Material | amount | volume | time | pressure | ratio | crude product, % |
| 1 | 2, 30 mg | PtO₂, | EtOH | 5 hr | 25° C. | 57.1:42.9 | 98.6 |
| | | 5 mg | 3 mL | | 3.5 atm | | |
| 2 | 5, 30 mg | PtO₂, | EtOH | 5 hr | 25° C. | 53.7:46.3 | 97.4 |
| | | 5 mg | 3 mL | | 3.5 atm | | |
| 3 | 2, 30 mg | PtO₂, | H₂O—MeOH | 18 hr | 25° C. | 60.3:39.7 | 96.2 |
| | | 5 mg | 90:10 | | 3.5 atm | | |
| | | | 3 mL | | | | |
| 4 | 5, 30 mg | PtO₂, | H₂O—MeOH | 18 hr | 25° C. | 58.4:41.6 | 94.2 |
| | | 5 mg | 90:10 | | 3.5 atm | | |
| | | | 3 mL | | | | |
| 5 | 2, 30 mg | PtO₂, | EtOAc, | 5 hr | 25° C. | 50.3:49.7 | 96.7 |
| | | 5 mg | 3 mL | | 3.5 atm | | |
| 6 | 5, 30 mg | PtO₂, | EtOAc, | 5 hr | 25° C. | 50.5:49:5 | 92.1 |
| | | 5 mg | 3 mL | | 3.5 atm | | |
| 7 | 2, 1 g | PtO₂, | EtOAc, | 18 hr | 25° C. | 53.6:46.4 | 96.7 |
| | | 5 mg | 10 mL | | 3.5 atm | | |
| 8 | 5, 1 g | PtO₂, | EtOAc, | 18 hr | 25° C. | 50.7:49.3 | 94.1 |
| | | 5 mg | 10 mL | | 3.5 atm | | |
| 9 | 2, 1 g | PtO₂, | EtOH,, | 18 hr | 25° C. | 58.5:41.5 | 95.9 |
| | | 5 mg | 10 mL | | 3.5 atm | | |
| 10 | 5, 1 g | PtO₂, | EtOH, | 18 hr | 25° C. | 54.9:45.1 | 93.8 |
| | | 5 mg | 10 mL | | 3.5 atm | | |
| 11 | 2, 1 g | PtO₂, | H₂O—MeOH | 18 hr | 25° C. | 61.7:38.3 | 94.6 |
| | | 5 mg | 90:10, | | 3.5 atm | | |
| | | | 10 mL | | | | |
| 12 | 5, 1 g | PtO₂, | H₂O—MeOH | 18 hr | 25° C. | 60.2:39.8 | 93.9 |
| | | 5 mg | 90:10, | | 3.5 atm | | |
| | | | 10 mL | | | | |

### EXAMPLES 13-20

Hydrogenations were carried out as in examples 1-12, except catalyst used was 5% palladium on carbon (Pd/C). The typical results are summarized in the Table 2.

TABLE 2

| | | | | | | | Menthol + |
|---|---|---|---|---|---|---|---|
| | | | Solvent | | Temperature, | Menthol | Isomenthol |
| Example | Starting | Catalyst, | and | Reaction | ° C., and | to Isomenthol | in crude |
| No. | Material | amount | volume | time | pressure | ratio | product, % |
| 13 | 2, 30 mg | 10% Pd/C | H₂O—MeOH | 12 hr | 25° C. | 73.6:26.4 | 96.0 |
| | | 50 mg | 90:10, | | 3.5 atm | | |
| | | | 3 mL | | | | |
| 14 | 5, 30 mg | 10% Pd/C | H₂O—MeOH | 12 hr | 25° C. | 70.7:29.3 | 92.4 |
| | | 50 mg | 90:10, | | 3.5 atm | | |
| | | | 3 mL | | | | |
| 15 | 2, 30 mg | 10% Pd/C | EtOH, | 6 hr | 25° C. | 74.6:25.4 | 97.2 |
| | | 50 mg | 3 mL | | 3.5 atm | | |
| 16 | 5, 30 mg | 10% Pd/C | EtOH, | 6 hr | 25° C. | 70.7:29.3 | 93.3 |
| | | 50 mg | 3 Ml | | 3.5 atm | | |
| 17 | 2, 30 mg | 10% Pd/C | EtOAc, | 6 hr | 25° C. | 70.1:29.9 | 95.3 |
| | | 50 mg | 3 mL | | 3.5 atm | | |
| 18 | 5, 30 mg | 10% Pd/C | EtOAc, | 6 hr | 25° C. | 69.4:30:6 | 90.4 |
| | | 50 mg | 3 mL | | 3.5 atm | | |
| 19 | 2, 1 g | 10% Pd/C | EtOH, | 24 hr | 25° C. | 73.5:26.5 | 93.8 |
| | | 7.5 mg | 10 mL | | 3.5 atm | | |
| 20 | 5, 1 g | 10% Pd/C | EtOH, | 24 hr | 25° C. | 70.3:29:7 | 92.7 |
| | | 7.5 mg | 10 mL | | 3.5 atm | | |

### EXAMPLES 21-33

The hydrogenations were carried out as in examples 1-20, except that catalyst used was 5% Palladium oxide on calcium carbonate. Typical results are summarized in the Table 3.

TABLE 3

| | | | | | Temperature, | Menthol | Menthol + |
|---|---|---|---|---|---|---|---|
| Example | Starting | Catalyst, | Solvent and | Reaction | ° C., and | to Isomenthol | isomenthol in |
| No. | Material | amount | volume | time | pressure | ratio | crude product, % |
| 21 | 2, 30 mg | 5% PdO/ | EtOAc, | 3 h | 25° C., | 72.2:27.8 | 94.6 |
| | | CaCO₃ | 3 mL | | 1 atm | | |
| | | 50 mg | | | | | |
| 22 | 2, 30 mg | 5% PdO/ | H₂O—MeOH | 3 h | 25° C., | 80.1:19.9 | 97.5 |
| | | CaCO₃ | 90:10, | | 1 atm | | |
| | | 50 mg | 3 mL | | | | |
| 23 | 2, 30 mg | 5% PdO/ | EtOH, | 3 h | 25° C., | 85.5:14.5 | 96.4 |
| | | CaCO₃ | 3 mL | | 1 atm | | |
| | | 50 mg | | | | | |
| 24 | 5, 30 mg | 5% PdO/ | EtOAc, | 3 h | 25° C., | 70.4:29.6 | 91.3 |
| | | CaCO₃ | 3 mL | | 1 atm | | |
| | | 50 mg | | | | | |
| 25 | 5, 30 mg | 5% PdO/ | H₂O—MeOH | 3 h | 25° C., | 79.0:21.0 | 93.0 |
| | | CaCO₃ | 90:10, | | 1 atm | | |
| | | 50 mg | 3 mL | | | | |
| 26 | 2, 30 mg | 5% PdO/ | EtOH, | 3 h | 25° C., | 82.5:17.5 | 94.4 |
| | | CaCO₃ | 3 mL | | 1 atm | | |
| | | 50 mg | | | | | |
| 27 | 5, 30 mg | 5% PdO/ | EtOH, | 3 h | 25° C., | 70.1:29.9 | 87.6 |
| | | CaCO₃ | 3 mL | | 1 atm | | |
| | | 50 mg | | | | | |
| | | 0.02 mL | | | | | |
| | | TEMED* | | | | | |
| 28 | 2, 30 mg | 5% PdO/ | H₂O—MeOH | 18 h | 4° C., | 84.8:15.2 | 96.0 |
| | | CaCO₃ | 90:10, | | 1 atm | | |
| | | 50 mg | 3 mL | | | | |
| 29 | 5, 30 mg | 5% PdO/ | H₂O—MeOH | 18 h | 4° C., | 77.0:23.0 | 94.3 |
| | | CaCO₃ | 90:10, | | 1 atm | | |
| | | 50 mg | 3 mL | | | | |
| 30 | 2, 30 mg | 5% PdO/ | EtOH, | 18 h | 4° C., | 81.8:18.2 | 96.9 |
| | | CaCO₃ | 3 mL | | 1 atm | | |
| | | 50 mg | | | | | |
| 31 | 5, 30 mg | 5% PdO/ | EtOH, | 18 h | 4° C., | 78.1:21.9 | 94.0 |
| | | CaCO₃ | 3 mL | | 1 atm | | |
| | | 50 mg | | | | | |
| 32 | 2, 1 g | 5% PdO/ | EtOH, | 18 h | 25° C., | 83.6:16.4 | 97.4 |
| | | CaCO₃ | 10 mL | | 3.5 atm | | |
| | | 7.5 mg | | | | | |
| 33 | 5, 1 g | 5% PdO/ | EtOH, | 18 h | 25° C., | 76.4:23.7 | 93.6 |
| | | CaCO₃ | 10 mL | | 3.5 atm | | |
| | | 7.5 mg | | | | | |

*TEMED - N,N′-tetramethylethylene-1,2-diamine.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for making (−)menthol, the method comprising:
a) providing starting material comprising 4R(+)-1-menthene having formula (1): 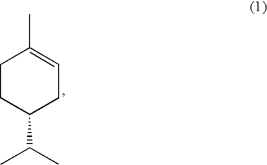
b) oxidizing the starting material in the presence of a catalyst comprising at least one polypeptide capable of hydroxylating at least one enantiomer of 1-menthene or limonene at an allylic carbon C(3), thereby forming a hydroxylated menthene product comprising at least 50% of the trans-piperitol having formula (2): 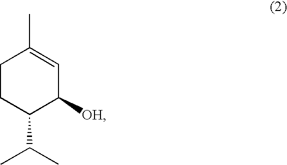
c) hydrogenating the trans-piperitol of formula (2) in the presence of a catalyst to form (−)-menthol having at least about 62% chemical purity, and at least about 90% enantiomeric excess, said (−)menthol represented by formula (3):

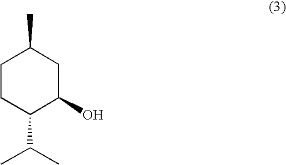

2. The method of claim 1, wherein 4R(+)-menthene in the starting material has optical purity of at least about 90%.

3. The method of claim 1, wherein 4R(+)-1-menthene in the starting material is derived from partial hydrogenation of orange or other citrus oil comprising at least about 90% of 4R(+)-limonene.

4. The method of claim 1, wherein the starting material comprises a mixture of 4R(+)-1-menthene and 4R(+)limonene in the ratio between about 1:100 to about 100:1.

5. The method of claim 1, wherein the polypeptide has limonene-3-hydroxylase activity.

6. The method of claim 1, wherein the polypeptide is encoded by nucleic acids isolated from at least one species of plant of genera selected from Lamiaceae, Umbelliferae, Asteraceae, Rutaceae, Rosaceae, Myrtaceae, Gramineae, Geranaceae.

7. The method of claim 1, wherein the polypeptide is encoded by nucleic acids isolated from at least one species of fungi or bacteria.

8. The method of claim 7, wherein species of fungi or bacteria are selected from Hormonema, Aspergillus, Pseudomonas, Rhodococcus, Bacillus.

9. The method of claim 1, wherein the oxidation step b) is carried out by at least one polypeptide encoded by modified nucleic acids, and wherein modifications of nucleic acids have been obtained by:
a) at least one method selected from mutagenesis, recombination, gene shuffling, combinatorial gene synthesis, and
b) at least one screening method allowing for detection of trans-piperitol (2) or trans-isopiperitenol (5) in the presence of other oxygenated menthene or limonene derivatives.

10. The method of claim 1, wherein the oxidation step b) is carried out by at least one polypeptide encoded by nucleic acids isolated from bacteria or fungi capable of growth on at least one compound selected from camphor, borneol, isoborneol,1,8-cineole, 1,4-cineole, fenchone, fenchol, terpineol, terpin hydrate and mixtures thereof.

11. The method of claim 1, wherein the oxidation step b) is carried out using hemoprotein enzyme as catalyst and hydrogen peroxide.

12. The method of claim 1, wherein the oxidation step b) is carried out in the presence of water-immiscible liquid organic solvent comprising 4R(+)-1-menthene, wherein said organic solvent to water ratio is at least about 1:50 by volume.

13. The method of claim 1, wherein the biological oxidation step b) is carried out by a polypeptide having regioselectivity for 3-trans-hydroxylation of R(+)-1-menthene (1) or 4R(+)-limonene (4) of at least about 75%.

14. The method of claim 1, wherein the biological oxidation step b) is carried out by a polypeptide having regioselectivity for 3-trans-hydroxylation of R(+)-1-menthene (1) or 4R(+)-limonene (4) of at least about 90%.

15. The method of claim 1, wherein the hydrogenation step c) is carried out using a catalyst selected from the group comprising platinum, palladium, palladium oxide, combinations thereof, and salts thereof.

16. The method of claim 15 wherein the catalyst comprises a substantially insoluble inorganic support selected from at least one salt of alkali earth metal and at least one acid, said acid being selected from the group comprising carbonic, phosphoric, and sulfric and mixtures thereof, or substantially insoluble support selected from silica, alumina, titania, zirconia, carbon, or mixtures thereof.

17. The method of claim 1, wherein the hydrogenation step c) is carried out using a catalyst comprising at least one of palladium or palladium oxide, salts thereof, and calcium carbonate.

18. The method of claim 1, wherein the hydrogenation step c) is carried out in the absence of solvent or in the presence of at least one solvent selected from the group comprising water, alcohol or glycol or polyol, said alcohol or glycol or polyol having linear or branched or cyclic alkyl or alkyloxyalkyl chain having from 1 to 20 carbon atoms.

19. The method of claim 1, wherein biological oxidation step b) is carried out using a monooxygenase enzyme in an electrochemical reactor.

20. A method for making (−)menthol, the method comprising:
a) providing starting material comprising 4R(+)-limonene having formula (4): 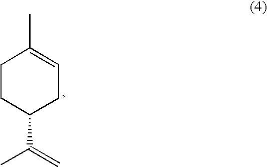
b) oxidizing the starting material in a presence of catalyst comprising at least one polypeptide capable of trans-hydroxylation of at least one enantiomer of 1-menthene or limonene at an allylic carbon C(3), thereby forming hydroxylated menthene product comprising at least about 75% of the trans-isopiperitenol having formula (5) 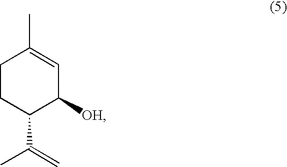
c) hydrogenating the trans-isopiperitenol of formula (5) in the presence of a catalyst, wherein hydrogenation selectivity of the said catalyst provides for (−)-menthol formation with a ratio of (−)menthol to (−)isomenthol of at least about 70:30, thereby forming (−)-menthol having at least about 70% chemical purity, and at least about 90% enantiomeric excess, said (−)menthol represented by formula (3):

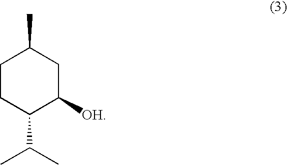

21. The method of claim 20, wherein 4R(+)-limonene in the starting material has optical purity of at least about 90%.

22. The method of claim 20, wherein the starting material comprises essential oil of orange or other citrus fruit, said oil containing at least about 90% of 4R(+)-limonene.

23. The method of claim 20, wherein the starting material comprises a mixture of 4R(+)-1-menthene and 4R(+)limonene in the ratio between about 1:100 to about 100:1.

24. The method of claim 20, wherein the polypeptide has R(+)-1-menthene 3-monooxygenase activity.

25. The method of claim 20, wherein the polypeptide is encoded by nucleic acids isolated from at least one species of plant of genera selected from Lamiaceae, Umbelliferae, Asteraceae, Rutaceae, Rosaceae, Myrtaceae, Gramineae, Geranaceae.

26. The method of claim 20, wherein the polypeptide is encoded by nucleic acids isolated from at least one species of fungi or bacteria.

27. The method of claim 26, wherein species of fungi or bacteria are selected from Hornonema, Aspergillus, Pseudomonas, Rhodococcus, Bacillus.

28. The method of claim 20, wherein the oxidation step b) is carried out by at least one polypeptide encoded by modified nucleic acids wherein modifications of nucleic acids have been obtained by using:
a) at least one method selected from mutagenesis, recombination, gene shuffling, combinatorial gene synthesis, and
b) at least one screening method allowing for detection of trans-piperitol (2) or trans-isopiperitenol (5) in the presence of other oxygenated menthene or limonene derivatives.

29. The method of claim 20, wherein the oxidation step b) is carried out by at least one monooxygenase encoded by nucleic acids isolated from bacteria or fungi capable of growth on at least one compound selected from camphor, borneol, isoborneol, 1,8-cineole, 1,4-cineole, fenchone, fenchol, terpineol, terpin hydrate and mixtures thereof.

30. The method of claim 20, wherein the oxidation step b) is carried out using hemoprotein enzyme as catalyst and hydrogen peroxide.

31. The method of claim 20, wherein the oxidation step b) is carried out in the presence of water-immiscible liquid organic solvent comprising 4R(+)-limonene wherein said organic solvent to water ratio is at least about 1:50 by volume.

32. The method of claim 20, wherein the biological oxidation step b) is carried out by monooxygenase having regioselectivity for 3-trans-hydroxylation of 4R(+)-limonene (4) or R(+)-1-menthene (1) of at least about 75%.

33. The method of claim 20, wherein the biological oxidation step b) is carried out by monooxygenase having regioselectivity for 3-trans-hydroxylation of 4R(+)-limonene (4) or of R(+)-1-menthene (1) of at least about 90%.

34. The method of claim 20, wherein the hydrogenation step c) is carried out using hydrogenation catalyst selected from the group consisting of palladium, palladium oxide, combinations thereof and salts thereof.

35. The method of claim 34, wherein the catalyst comprises a substantially insoluble inorganic support selected from at least one salt of an alkali earth metal and at least one acid, said acid being selected from the group comprising carbonic, phosphoric, or sulfuric, and mixtures thereof, or substantially insoluble support selected from silica, alumina, titania, zirconia, carbon, or mixtures thereof.

36. The method of claim 20, wherein the hydrogenation step c) is carried out using a catalyst comprising at least one of palladium and palladium oxide, salts thereof, and calcium carbonate.

37. The method of claim 20, wherein the hydrogenation step c) is carried out in the absence of solvent or in the presence of at least one solvent selected from the group consisting of water, of alcohol or glycol or polyol, said alcohol or glycol or polyol having linear or branched or cyclic alkyl or alkyloxyalkyl chain having from 1 to 20 carbon atoms.

38. The method of claim 1, wherein the oxidation step b) is carried out using a monooxygenase enzyme in an electrochemical reactor.

39. A method for making (−)menthol, comprising:
a) biologically oxidizing a starting material comprising 4R(+)-1-menthene having formula(1): 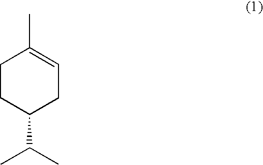
in the presence of at least one polypeptide to form a product comprising at least 50% of the trans-piperitol having formula (2): 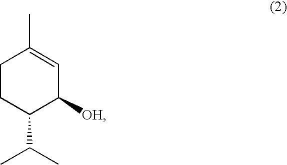
b) hydrogenating the trans-piperitol of formula (2) in the presence of a catalyst to form the (−)-menthol of formula (3), wherein the (−) menthol has a chemical purity of at least about 62%, and wherein the the (−)-menthol is formed in at least about 90% enantiomeric excess:

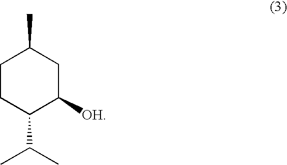

40. The method of claim 39, wherein the (−)-menthol has a chemical purity of at least about 70%.

41. The method of claim 39, wherein the (−)-menthol has a chemical purity of at least about 72%.

42. The method of claim 39, wherein the catalyst is selected from the group consisting of platinum, palladium, palladium oxide, combinations thereof and salts thereof.

43. The method of claim 42, wherein the catalyst is selected from at least one of palladium and palladium oxide immobilized on calcium carbonate.
